SEQUENCE LISTING (31 sequences)

SEQ ID NO: 1 - protein, 7 aa, artificial sequence
  autolytic site 1 of calpain 3
NMTYGTS

SEQ ID NO: 2 - protein, 7 aa, Artificial Sequence
  autolytic site 2 of calpain 3
NMDNSLL

SEQ ID NO: 3 - protein, 7 aa, artificial sequence
  autolytic site 3 of calpain 3
PVQYETR

SEQ ID NO: 4 - protein, 12 aa, artificial sequence
  autolytic site of human calpain 3
VAPRTAAEPRSP

SEQ ID NO: 5 - protein, 12 aa, artificial sequence
  autolytic site of human calpain 3
QSKATEAGGGNP

SEQ ID NO: 6 - protein, 12 aa, artificial sequence
  autolytic site of murine calpain 3
VAPRTGAEPRSP

SEQ ID NO: 7 - protein, 12 aa, artificial sequence
  autolytic site of murine calpain 3
QGKTTEAGGGHP

SEQ ID NO: 8 - protein, 10 aa, artificial sequence
  autolytic site of rodent Lp82
NPYLLPGFFC

SEQ ID NO: 9 - protein, 9 aa, artificial sequence
  autolytic site of rodent Lp82
TISVDRPVP

SEQ ID NO: 10 - protein, 13 aa, artificial sequence
  human calpastatin
REVTIPPKYRELL

SEQ ID NO: 11 - protein, 13 aa, artificial sequence
  mouse and rat calpastatin
KEGTIPPEYRKLL

SEQ ID NO: 12 - protein, 14 aa, artificial sequence
  human alpha-A-crystalline
PVSREEKPTSAPSS

SEQ ID NO: 13 - protein, 14 aa, artificial sequence
  mouse and rat alpha-A-crystalline
PVSREEKPSSAPSS

SEQ ID NO: 14 - protein, 11 aa, artificial sequence
  human talin
KSTVLQQQYNR

SEQ ID NO: 15 - DNA, 20 bp, artificial sequence
  SFp94S1.a
ccggaagtggcacgaacatg

SEQ ID NO: 16 - DNA, 20 bp, artificial sequence
  SGp94S2.a
ccggaagtggcgtgagaaat

SEQ ID NO: 17 - DNA, 20 bp, artificial sequence
  SGp94S3.a
ccggaagtggcattgttccc

SEQ ID NO: 18 - protein, 62 aa, artificial sequence
  This is a partial sequence of calpain 3.
DGTNMTYGTSPSGLNMGELIARMVRNMDNSLLQDSDLDPRGSDERPTRTIIPVQYETRMACG

SEQ ID NO: 19 - DNA, 97 bp, artificial sequence
  pTOM
tacaagtccggactcagatctcgagctcaagcttcgaattctgcagtcgacggtaccgcgggcccgggatccaccggtcgccaccatggtgagcaag

SEQ ID NO: 20 - protein, 32 aa, artificial sequence
  Sequence A
YKSGLRSRAQASNSAVDGTAGPGSTGRHHGEQ

SEQ ID NO: 21 - protein, 19 aa, artificial sequence
  Sequence B
YKSGLRSRGDPPVATMVSK

SEQ ID NO: 22 - DNA, 92 bp, artificial sequence
  pTOM cloning Site
gctgtacaagtccggactcagatctcgagctcaagcttcgaattctgcagtcgacggtaccgcgggcccgggatccaccggtcgccaccatg

SEQ ID NO: 23 - DNA, 53 bp, artificial sequence
  Bsp-p94S1.a
ccggaagtggcacgaacatgacttacgggacctctccttctggttcagggtcg

SEQ ID NO: 24 - DNA, 53 bp, Artificial sequence
  Bam-p94S1.m
ttcaccgtgcttgtactgaatgccctggagaggaagaccaagtcccagcctag

SEQ ID NO: 25 - protein, 18 aa, artificial sequence
  Calpain 3 Site 1
SGSGTNMTYGTSPSGSGS

SEQ ID NO: 26 - DNA, 56 bp, artificial sequence
  Bsp-p94S2.a
ccggaagtggcgtgagaaatatggataactcgctgctcagagactcagggagtggg

SEQ ID NO: 27 - DNA, 56 bp, artificial sequence
  Bam-p94S2.m
ttcaccgcactctttatacctattgagcgacgagtctctgagtccctcacccctag

SEQ ID NO: 28 - protein, 19 aa, artificial sequence
  Calpain 3 site 2
SGSGVRNMDNSLLRDSGSG

SEQ ID NO: 29 - DNA, 56 bp, artificial sequence
  Bsp-p94S3.a
ccggaagtggcattgttcccgtgcagtatgaaacaagaatggcctgtgggagtggg

SEQ ID NO: 30 - DNA, 56 bp, artificial sequence
  Bam-p94S3.m
ttcaccgtaacaagggcacgtcatactttgttcttaccggacaccctcacccctag

SEQ ID NO: 31 - protein, 19 aa, artificial sequence
  Calpain 3 site 3
SGSGIVPVQYETRMACGSG

(19) United States
(12) Patent Application Publication
(10) Pub. No.: US 2004/0180395 A1
(43) Pub. Date: Sep. 16, 2004

(54) Method for detecting calpain3 activity in a biological sample and peptides for implementing said method

(76) Inventors: Isabelle Richard, Corbeil-Essonnes (FR); Guillaume Sillon, Sceaux (FR)

Correspondence Address: Sanford E Warren Jr, Warren & Kennedy, Suite 910, 6565 MacArthur Boulevard, Irving, TX, 75039-2461, US

(21) Appl. No.: 10/432,688

(22) PCT Filed: Jun. 24, 2002
(86) PCT No.: PCT/FR02/02178

(30) Foreign Application Priority Data
Jun. 29, 2001 (FR) 01/08614

(51) Int. Cl. C12Q 1/37; C07K 7/08

(52) U.S. Cl. 435/23; 530/409; 530/328; 530/329

(57) ABSTRACT

Peptide coupled with at least one fluorogenic or colorogenic reporter molecule, characterized in that it contains at least one amino acid sequence able to be cleaved by calpain 3.

Method for detecting, in vitro, the activity of calpain 3 in a biological sample.

The invention relates to a method for detecting calpain 3 activity in a biological sample consisting either of cells or of cell lines or of tissues. It also relates to the peptides intended to be used in said method. The invention also relates to the use of said peptides for the in vitro diagnosis of limb-girdle muscular dystrophy type 2A (LGMD 2A). Moreover, it relates to a method for analysing the efficiency of transfer of the calpain 3 gene into animal or human cells in vitro, but also into animals in vivo. It also relates to a method for screening for caipain 3-inhibiting or -activating substances. Finally, the method for detecting calpain 3 activity constitutes a means for studying the function of calpain 3.

Calpains are a family of calcium-activatable, non-lysosomal cysteine proteases [Sorimachi, 1997]. This family comprises, at the current time, 11 members, including 2 ubiquitous proteins. The physiological functions of calpains are still largely unknown. As regulatory-type proteases, they should probably regulate important cell functions. In particular, ubiquitous calpains are involved in apoptosis (Squier, 1994], myogenic differentiation [Kwak, 1993], and cell division and fusion [Yamaguchi, 1994]; [Schollmeyer, 1986]; [Balcerzak, 1995].

Calpain 3, also known as p94, is a calcium-dependent cysteine enzyme belonging to the family of calpains expressed specifically in skeletal muscle [Sorimachi, 1989]. It is involved in an autosomal recessive genetic disease called limb-girdle muscular dystrophy type 2A [Richard, 1995]. This myopathy is characterized by an atrophy and a progressive weakness of the muscles of the pelvic and shoulder girdles, and an appearance of necrosis-regeneration on muscle biopsies [Fardeau, 1996]. The gene, located on chromosome 15 in humans, encodes a 3.5 kb transcript, which itself encodes a 94 kDa protein. It has been demonstrated that calpain 3 can bind to titin, an elastic protein of the sarcomere, via the IS₂ region, and that it undergoes autolytic degradation immediately after translation when it is expressed in Cos7 cells [Sorimachi, 1993] [Sorimachi, 1995]. This region comprises a nuclear localization signal implying that calpain can be located either in the cytoplasm or in the nucleus. It has, moreover, been shown that calpain 3 is underexpressed in transgenic mice overexpressing interleukin 6, these mice exhibiting muscular atrophy. Calpain 3 also appears to be underexpressed in various conditions comprising a muscle-atrophy component (cachexia, etc).

With the current state of knowledge, it is therefore known that LGMD 2A is due to mutations which appear on the calpain 3 gene, mutations which lead to inhibition of proteolysis of the calpain 3 present in skeletal muscle. The clinical diagnosis of LGMD 2A is very difficult since the patients present clinical signs similar to at least about ten other pathological conditions. As regards the molecular diagnosis, it may be carried out according to various methods.

The first possibility is to perform a mutation search on the calpain gene. However, such a technique is very laborious since the gene is relatively large, a large number of different mutations therefore exist, and there is no preferential mutation.

Another technique consists in detecting the presence of the protein using specific antibodies. However, even if calpain 3 is present in the sample, this does not mean that the individual is not ill since the mutation on the calpain gene may mean that the protein is present but that the phenomenon of autolysis does not exist. On the other hand, if calpain is absent or decreased, it may involve a secondary calpainopathy due to mutations on a gene other than that of calpain.

In other words, the problem which the invention proposes to solve is not so much to detect the presence of calpain 3 in a biological sample, but rather that of detecting its activity.

Guttmann et al. describe, in the document “Methods in molecular biology” vol 144, ch 18, a method for measuring ubiquitous calpain activity consisting in bringing a purified calpain into contact with a nonspecific fluorescent peptide (Succ-LLVY-AMC), and then measuring the variation of fluorescence which appears when the molecule is cleaved. In a second method, the calpain is brought into contact with the whole TAU protein, and then Western blotting is carried out. That document does not in any way relate specifically to calpain 3. In addition, the substrates are either nonspecific (substrate of m-calpain and μ-calpain), or whole proteins.

The problem which the invention proposes to solve is to develop a novel substrate specific to calpain 3, which may be used to detect calpain 3 activity in a biological sample.

Consequently the invention relates first of all to a peptide coupled to at least one fluorogenic or colorogenic reporter molecule, said peptide being characterized in that it contains at least one amino acid sequence able to be cleaved by calpain 3 or an isoform of calpain 3.

The expression “isoform of calpain 3” denotes any protein produced by the calpain 3 gene, resulting from an alternative event of the alternative promoter or alternative splicing type.

In a first embodiment, and given the autolytic properties of calpain 3, the peptide of the invention advantageously contains at least one autolytic site of calpain 3 or of an isoform of calpain 3, the number of amino acids of which is less than 10.

In the remainder of the description and in the claims, the expression “autolytic site” denotes an amino acid sequence contained in calpain 3, or one of its isoforms, which can be cleaved by calpain 3, or one of its isoforms, into at least-two peptides, and also any derived sequence.

In practice, the amino acid sequence of the autolytic site of calpain 3 is chosen from the following sequences NMTYGTS (SEQ ID1), NMDNSLL (SEQ ID2) and PVQYETR (SEQ ID3) named, in the remainder of the description, site 1, site 2 and site 3, respectively. These sites are identical in all species for which the calpain 3 sequence is known to date (humans, mice, rats).

The amino acid sequence of the autolytic site of calpain 3 can also be chosen from the following human sequences VAPRTA AEPRSP (SEQ ID4), QSKATE AGGGNP (SEQ IDS), and the following murine sequences VAPRTG AEPRSP (SEQ ID6), QGKTTE AGGGHP (SEQ ID7).

In the embodiment according to which the peptide of the invention contains at least one autolytic site of an isoform of calpain 3, the amino acid sequence of the autolytic site originates from an isoform of calpain 3 named Lp82, present in the rodent, rats and mice, and is chosen from the following amino acid sequences: NPYLLPGFFC (SEQ ID8) and TISVDRPVP (SEQ ID9).

In a second embodiment, the amino acid sequence which can be cleaved by calpain 3 or an isoform of calpain 3 originates from a substrate protein such as, for example, calpastatin, filamin, talin, ubiquitous calpains, crystalline, heat shock protein, etc.

Advantageously, the substrate proteins have the following sequences:

REVTIPPKYRELL (SEQ ID10) (human calpastatin)

KEGTIPPEYRKLL (SEQ ID11) (mouse and rat calpastatin)

PVSREEKPTSAPSS (SEQ ID12) (human alpha-A-crystalline)

PVSREEKPSSAPSS (SEQ ID13) (mouse and rat alpha-A-crystalline)

KSTVLQQQYNR (SEQ ID14) (human talin)

Sequence published under the accession number XP 045856 (human filamin 2)

Sequence published under the accession number P 10809 (human heat shock protein 60)

Sequence published under the accession number NP 004355 (human c/EBP beta)

Sequence published under the accession number P 17655 (human m-calpain)

In a third embodiment, the peptide containing a sequence which can be cleaved by calpain 3 or an isoform of calpain 3 is obtained by screening a peptide library with calpain 3 or an isoform of calpain 3.

In order to allow subsequent detection of calpain activity by colorimetry or fluorometry, the peptide of the invention, as already mentioned, is coupled to a colorogenic or fluorogenic reporter molecule.

When the reporter molecule is a colorogenic molecule, cleavage of the amino acid sequence by calpain 3 will be detected on a spectrophotometer by the appearance of a coloured compound. In practice, the coloured compound used is para-nitroanilide. It may also be thioesters.

When the peptide is coupled to a fluorogenic compound, the cleavage is detected by a change in fluorescent emission. In this case, and in practice, the fluorogenic compound used is 4-methyl-7-coumarylamide (MCA) or naphthylamide. Naphthylamide and 7-amino-3-fluoromethylcoumarylamide can be used as a colorogenic or fluorogenic substrate.

In another embodiment, the peptide which can be cleaved with calpain 3 is coupled at both its ends with two fluorogenic compounds, the cleavage being detected by a change in fluorescent emission of a first compound (donor molecule) due to the distancing, secondary to the cleavage, of a second compound (acceptor molecule) located on the other side of the peptide and which absorbs the fluorescence of the first when these molecules are close. This technique is well known under the name FRET (Fluorescence Resonance Energy Transfer) (Förster, 1948). More specifically, FRET is a physical phenomenon which can occur between two fluorescent molecules under certain conditions: the two molecules must be sufficiently close to one another (less than 100 Å) and the emission spectrum of one of the molecules (the donor molecule) must cover the excitation spectrum of the second molecule (the acceptor molecule). Thus, when the donor molecule is excited at its excitation wavelength, it reaches a higher level of energy. In a few picoseconds, part of the energy is dispersed in the medium in different forms (heat, etc). If the donor molecule is in an optimal orientation and in proximity to an acceptor molecule, its energy can be transferred to this acceptor molecule without the involvement of a photon or the need for a collision between the two molecules. The acceptor molecule is then excited and emits light at its own emission wavelength. When the peptide is cleaved, the energy is no longer absorbed and the donor molecule emits fluorescence. The increase in fluorescence therefore corresponds to a measurable activity of peptide cleavage. The fluorogenic reporter molecule may be a synthetic molecule obtained by chemical synthesis or a protein which allows emission of a fluorescent signal.

In a first embodiment, the peptide has, at each of its ends, a synthetic fluorogenic reporter molecule, respectively MCA (donor molecule) and Dnp (acceptor molecule).

In a second embodiment, the donor is 5-[(2′-amino-ethyl)amino]naphthalenesulphonic acid (EDANS) and the acceptor is 4-[[4′-(dimethylamino]phenyl]azobenzoic acid (DABCYL).

In all the above cases, the peptide of the invention coupled to the colorogenic or fluorogenic molecule is obtained by chemical synthesis.

As already mentioned, the reporter molecule may also be a protein which allows emission of a fluorescent signal. That being the case, and in an advantageous embodiment, the peptide has, at each of its ends, a mutated GFP.

GFP (Green Fluorescent Protein) is a 27 kDa protein which is synthesized by a Pacific jellyfish (Aequora Victoria) and which emits a green fluorescence when the animal is subjected to stress [Prasher, 1992]. It has been possible to purify this protein and to identify its gene. The sequence encoding GFP can be cloned in phase with sequences encoding very diverse proteins. GFP keeps its properties of fluorescence when it is linked to other proteins, which makes it easy to study many phenomena due to proteins to which GFP is linked, such as, for example, the study of cell migration or of migration of a protein inside the various cellular compartments. Certain mutations of GFP in amino acids forming the fluorophore or interacting with this fluorophore have made it possible to identify mutants having specific fluorescence characteristics [Ellenberg, 1999; Pollock, 1999]. In particular, the CFP, for Cyan Fluorescent Protein (excitation 440 nm; emission 480 nm) and YFP, for Yellow Fluorescent Protein (excitation 514 nm; emission 530 nm) proteins have been isolated. These two proteins can in theory be connected via a phenomenon of FRET. Effectively, there is an overlap between the emission spectrum of CFP (the donor molecule) and the excitation spectrum of YFP (the acceptor molecule).

When these two proteins are close enough to one another and when CFP is excited at its excitation wavelength, CFP can transfer its activation energy to YFP, which can then emit a light at 535 nm. The sequences corresponding to the autolytic site of calpain 3 mentioned above will therefore be cloned between the CFP and YFP sequences. This system will be used to detect calpain 3 by producing, in live cells, chimeric proteins in which CFP will be connected to YFP via a calpain 3-cleavable peptide.

The subject of the invention is also a DNA sequence encoding a peptide coupled to at least one fluorescent protein, said peptide containing at least one amino acid sequence able to be cleaved by calpain 3 or an isoform of calpain 3. In a preferred embodiment, the DNA sequence encodes the following peptides:

CFP-site 1-YFP;

CFP-site 2-YFP;

CFP-site 3-YFP

The invention also relates to a vector contained in said DNA sequence and a promoter for inducing expression of the DNA sequence in a host cell. Such a vector is, for example, the plasmid pTOM developed by the Applicant, directly derived from the plasmid described in [Vanderklish 2000]. It also relates to a host cell transformed with said vector.

The invention also relates to a method for detecting, in vitro, the activity of calpain 3 or of an isoform of calpain 3 in a biological sample, according to which: in a first step, said biological sample is brought into contact with the peptide described above, in a second step, the presence or absence of cleavage of said peptide by calpain 3 or an isoform of calpain 0.3 is detected by measuring the intensity of the calorimetric or fluorometric reaction.

The first step may assume various forms depending on whether the detection is carried out on a biological sample consisting of either live cells or of a cell extract, the cells being of animal or human origin, or of a tissue.

When the sample consists of live cells, the contact with the peptide may take place in two ways. In a first embodiment, the peptide is brought directly into contact with the cell in such a way that it must exhibit properties of sufficient permeability to penetrate into the cell. The permeability of the peptide will be determined as a function of the nature of the reporter molecule. In a second embodiment, the biological sample corresponds to host cells transfected with a vector encoding the DNA sequence corresponding to the peptide of the invention, the reporter molecules then corresponding to proteins which allow emission of a fluorescent signal.

When the sample is in the form of a cell extract, the peptide is simply brought into contact with said extract.

The detection of the activity may also take place, as already mentioned, directly on tissue sections. In practice, the biological sample (organ, part of an organ, for example muscle biopsies) is then taken and then rapidly frozen in isopentane cooled with liquid nitrogen. It is stored at −80° C. until use. Sections of 5 to 15 μm are prepared using a cryostat and placed on a glass slide. The sections are also stored at −80° C. if they are not used immediately. The detection of calpain activity can be carried out by depositing the peptide directly onto the slide.

In an advantageous embodiment, the peptide has, at each of its ends, a fluorogenic donor molecule and a fluorogenic acceptor molecule, respectively, the intensity of the fluorogenic reaction being determined by FRET. Thus, if the calpain is not active in the cells, a FRET phenomenon occurs. On the other hand, if the calpain is active, it cleaves the peptide and the FRET phenomenon is then decreased.

The method for detecting the activity of calpain 3 or of an isoform of calpain 3 finds an advantageous application for the in vitro diagnosis of LGMD 2A. Consequently, the invention also relates to the use of the detection method described above for the in vitro diagnosis of LGMD 2A.

The invention also relates to a method for screening for substances which inhibit or activate calpain 3 or an isoform of calpain 3. Said method may assume two different embodiments.

According to a first embodiment, the method consists:

in preparing a biological sample treated with said substance,

then in bringing said sample thus treated into contact with the peptide of the invention,

and in detecting the presence or absence of a calorimetric or fluorometric reaction, indicating respectively the presence or absence of a substance which activates or inhibits calpain 3 or an isoform of calpain 3.

The biological sample may be in the form of cells, of cell extracts or else of tissues. Preparation of the biological sample containing the peptide is then carried out by mixing the treated sample (cell, cell extract or tissue extract) with the peptide. In practice, the peptide has, at each of its ends, a fluorogenic donor molecule and a fluorogenic acceptor molecule, respectively, the intensity of the fluoro-genic reaction being determined by FRET.

According to a second embodiment, the method consists:

in preparing a biological sample containing the peptide according to the invention,

then in bringing said sample into contact with the substance to be identified,

and in detecting the presence or absence of a calorimetric or fluorometric reaction, indicating respectively the presence or absence of a substance which activates or inhibits calpain 3 or an isoform of calpain 3.

In this case, the biological sample consists of cells or cell lines transfected with a vector comprising the DNA sequence encoding the peptide of the invention, when the reporter molecule is of protein origin.

In an advantageous embodiment, the peptide has, at each of its ends, a fluorogenic donor molecule and fluoro-genic acceptor molecule, respectively, and the method consists in:

a/ preparing a biological sample containing said peptide,

b/ measuring the amount of FRET in the absence of the substance which activates or inhibits calpain 3 or an isoform of calpain 3,

c/ bringing the biological sample containing the peptide into contact with the substance which activates or inhibits calpain 3 or an isoform of calpain 3

d/ measuring the amount of FRET in the presence of the substance which activates or inhibits calpain 3 or an isoform of calpain 3,

e/ drawing the conclusion of the presence:

of an activating substance if the amount of FRET measured in b/ is greater than the amount of FRET measured in d/;

or of an inhibiting substance if the amount of FRET measured in b/ is equal to the amount of FRET measured in d/.

The subject of the invention is also a method for analysing the efficiency of transfer of the calpain 3 gene, consisting:

first of all in transfecting animal or human cells with a plasmid encoding calpain 3,

then in bringing the transfected cells into contact with the peptide of the invention, either in vitro or in vivo,

then in measuring the intensity of the calorimetric or fluorometric reaction.

In an advantageous embodiment, the peptide has, at each of its ends, a fluorogenic donor molecule and a fluorogenic acceptor molecule, respectively, the intensity of the fluorogenic reaction being determined by FRET.

The calpain 3 gene has been completely identified, and the transfection techniques have been precisely described in document EP 717110, such that further details of them will not be given.

The invention and the advantages which ensue therefrom will emerge more clearly from the following examples of implementation in support of the attached figures.

FIG. 1: Partial sequence of calpain 3; the position of the autolytic sites is indicated by the arrows; the sequences used in the peptides are underlined and are identical in all species for which the sequence of calpain 3 is known to date (humans, mice, monkeys, rats, bovines).

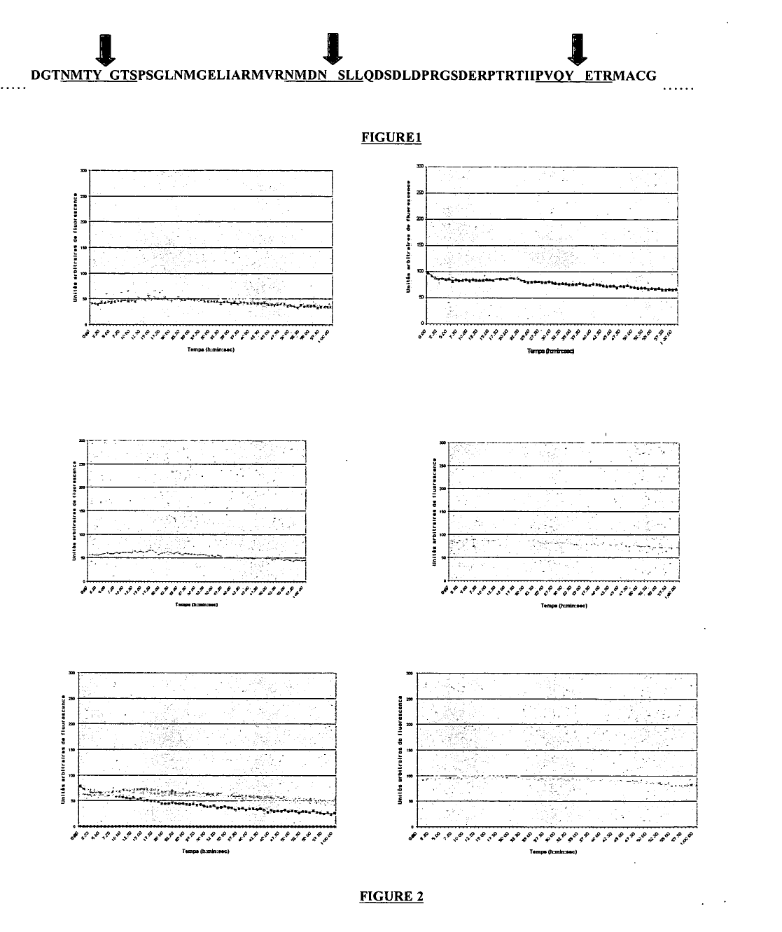

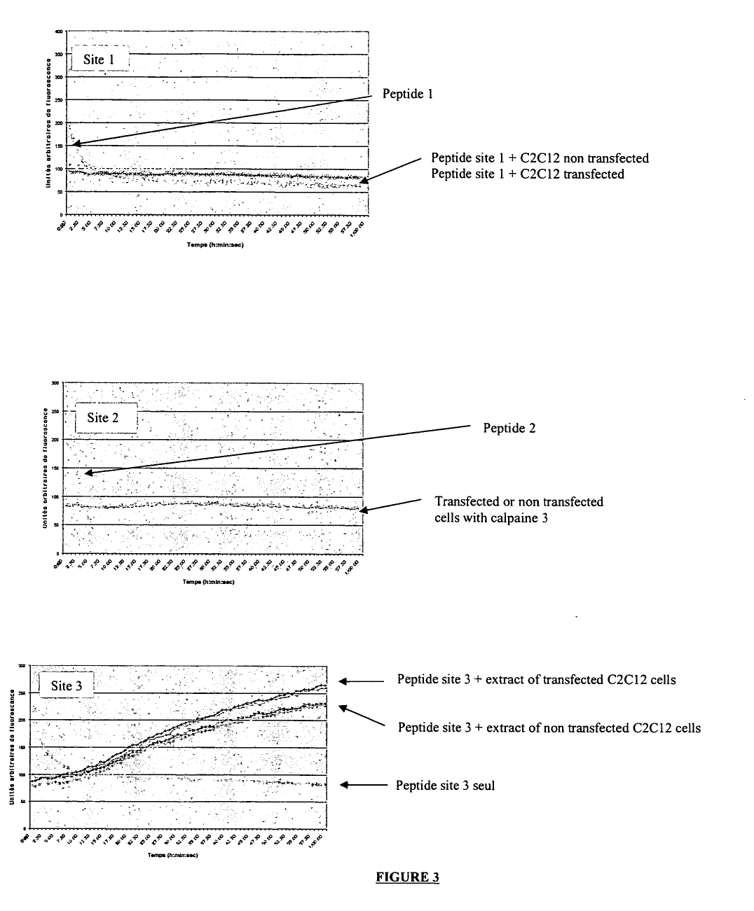

FIG. 2: Measurement over time of the activity of cleavage of the peptides corresponding to autolytic sites 1, 2 and 3 of calpain 3 (curves A, B and C, respectively) by recombinant calpains 1 (left-hand column) and 2 (right-hand column).

FIG. 3: Measurement over time of the activity of cleavage of the peptides corresponding to the autolytic sites calpain 3 by extracts of C2C12 cells transfected or not transfected with the plasmid encoding calpain 3.

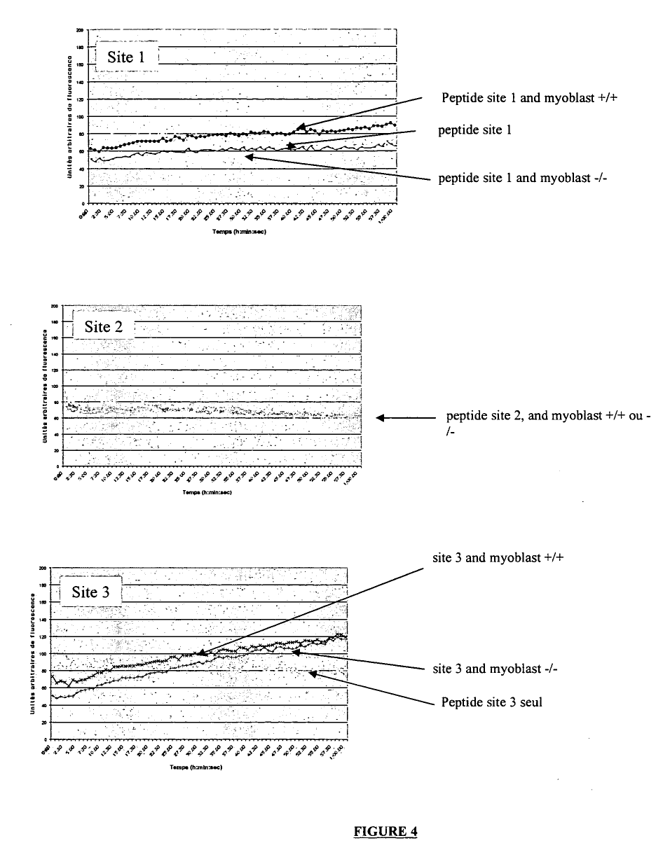

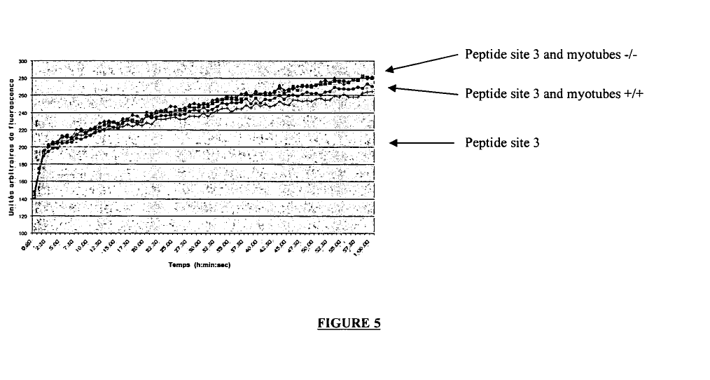

FIG. 4: Measurement over time of the activity of cleavage of the peptides corresponding to the autolytic sites of calpain 3 by extracts of normal (+/+) or calpain 3-deficient (−/−) mouse myoblasts.

FIG. 5: Measurement over time of the activity of cleavage of the peptide corresponding to autolytic site 3 of calpain 3 by extracts of normal (+/+) or calpain 3-deficient (−/−) mouse myotubes.

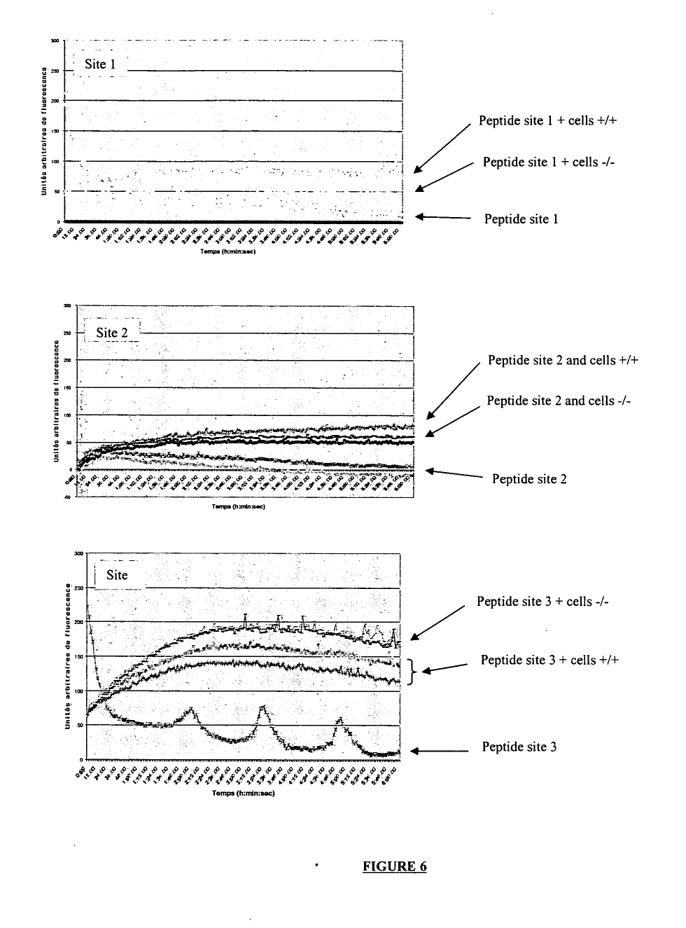

FIG. 6: Measurement over 6 hours of the activity of cleavage of the peptides corresponding to the autolytic sites of calpain 3 by extracts of normal (+/+) or calpain 3-deficient (−/−) mouse myoblasts.

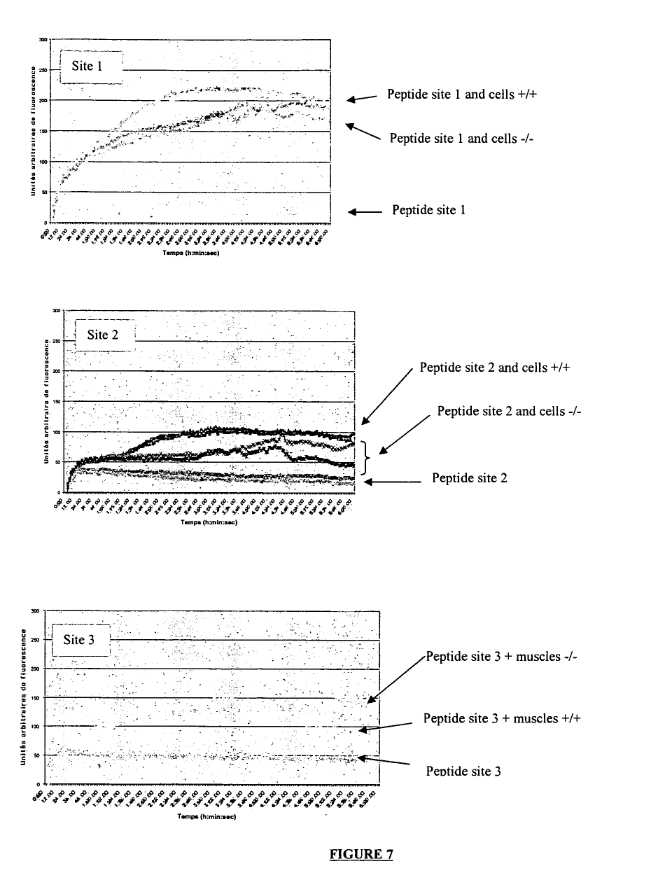

FIG. 7: Measurement over 6 hours of the activity of cleavage of the peptides corresponding to the autolytic sites of calpain 3 by extracts of normal (+/+) or calpain 3-deficient (−/−) mouse quadriceps.

FIG. 8: Portion of sequence of the plasmid pTOM. The amino acids in italics are part of the sequence of EYFP. The amino acids in bold are part of that of ECFP. The STOP codon of EYFP has been removed in the vector pTOM. The bases in italics and in bold show the phases of the EYFP and ECFP sequences, respectively. The bases underlined correspond to the fragment which was removed to construct the vector pTOMp. The protein sequences A and B correspond to the translation of the vector pTOM from the ATG of the coding sequence EYFP, respectively before and after removal of the double-stranded fragment located between the Ecl136II and SmaI restriction sites. In the sequence A, the sequences encoding EYFP and ECFP are not in phase. They are in phase in sequence B.

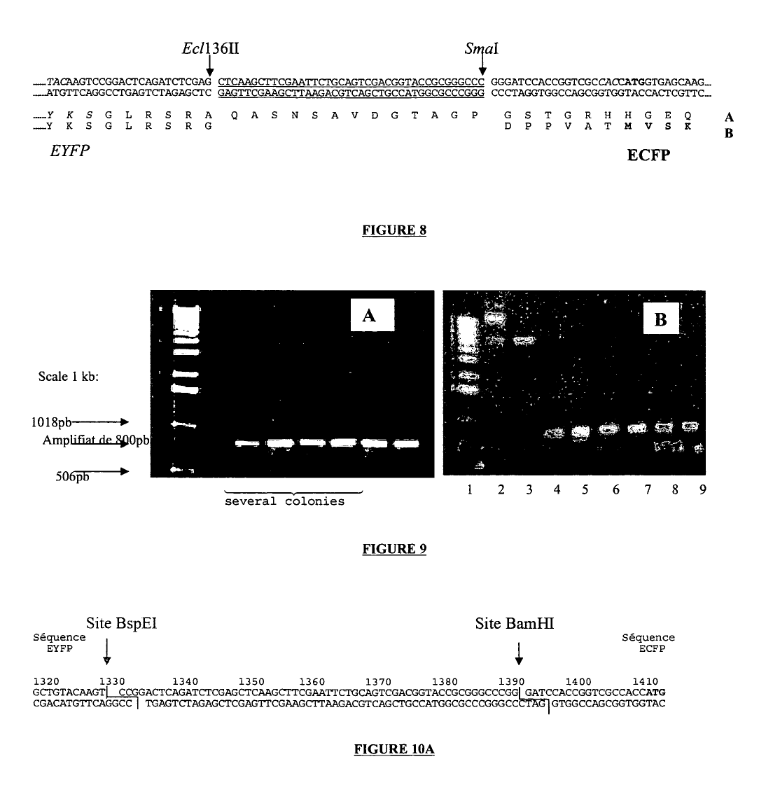

FIG. 9: A: Migration on an agarose gel of the products of PCR on colonies subcultured after trans-formation with pTOMp. The PCRs were carried out with the oligonucleotides midEYFP.a and midECFP.m. B: Migration of these PCR products after digestion with EcoRI; well No. 1: 1 kb ladder; No. 2: pTOM; No. 3: pTOM after digestion with EcoRI; No. 4 to No. 9: migration of the PCR products of gel A after digestion with EcoRI; they are always 800 bp in size.

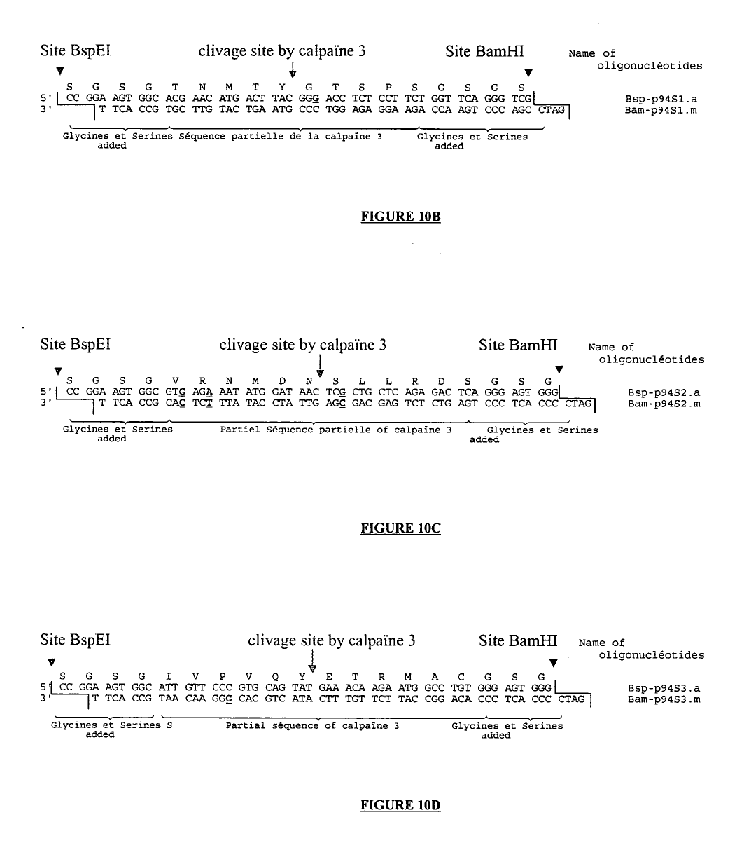

FIG. 10: Cloning site of the vector pTOM (10a) and sequence of the oligonucleotides encoding the autolytic sites of calpain 3 (10b, 10c, 10d). Each pair of complementary oligonucleotides is composed of an oligonucleotide whose name ends with “.a” and an oligonucleotide whose name ends with “.m”.

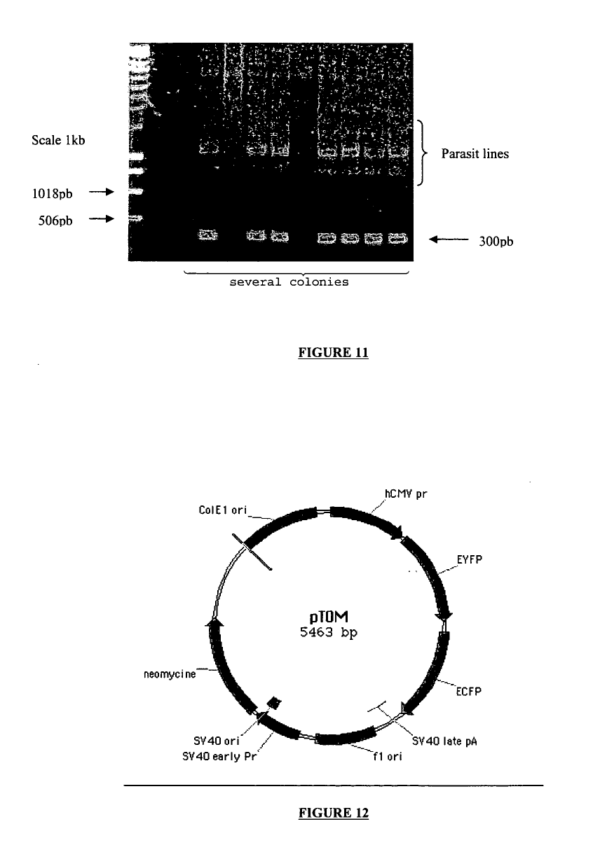

FIG. 11: Migration on an agarose gel of products of PCR on colonies subcultured after transformation with pTOM and the insert encoding autolytic site 2; the PCR was carried out with the oligonucleotide SGp94S2. a and midECFP.m.

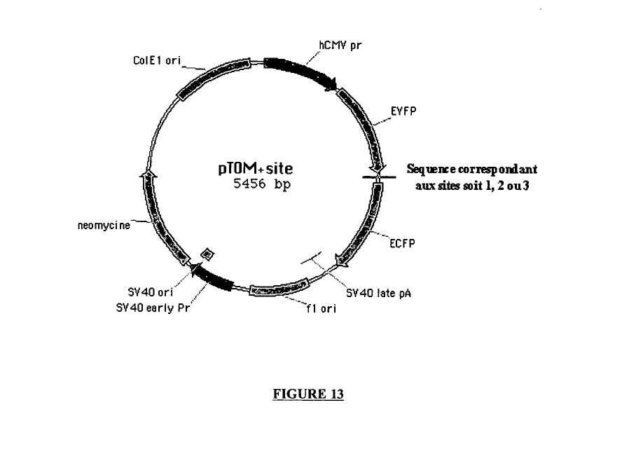

FIG. 12: Restriction map of plasmid pTOM.

FIG. 13: Restriction map of plasmids pTOMs1, pTOMs2 and TOMs3.

### I/ ASSAY OF ACTIVITY OF CALPAIN 3 USING PROTEIN EXTRACTS

The cleavage of synthetic peptides corresponding to the autolytic sites of calpain 3 (FIG. 1) makes it possible to detect its activity using a technique involving FRET.

### A/ Materials and Methods

1/ Fluorometer: SpectraMax Gemini XS Microplate fluoro-meter (molecular Devices).

2/ Sequence of the Fluorescent Peptides:

Mca-NMTYGTS-Dnp (site 1)

Mca-NMDNISLL-Dnp (site 2)

Mca-PVQYETR-Dnp (site 3)

3/ References of the commercial Enzymes and of the Inhibitors Used:

Calpain 1: human erythrocytes (Calbiochem)

Calpain 2: rat, recombinant, E. coli (Calbiochem)

Caspase 3: human, recombinant, E. coli (Calbiochem)

4/ Reaction Conditions:

Final volume of 200 μl; calcium (CaCl₂) 10 mM final concentration; fluorescent peptides 1.9 μM final concentration.

5/ Detection of Fluorescence:

The reaction is carried out at 37° C., either over one hour (with in this case measurement of fluorescence every 50 seconds) or over 6 hours (measurement every 2 minutes).

The medium is agitated between each measurement. The wavelengths used for detecting the fluorescence of the peptides are:

Excitation wavelengths of Mca: 325 nm.

Emission wavelengths of Dnp: 392 nm.

6/ Setting up of Buffers

Initially, the reaction conditions were set up, and in particular the composition of the reaction buffer. Effectively, detection of the fluorescence proved to be very sensitive. The first assays consisted in varying the various components of the buffer and their concentration. Various concentrations of NaCl were tested. The importance of having a low concentration of DMSO, in which the peptides are resuspended, was demonstrated. It was also shown that the presence of CHAPS in the reaction medium, and also BSA, enabled optimal detection of fluorescence. In order to be certain of activating calpain 3, the reactions were carried out at 37° C., in the presence of 10 mM of calcium: Effectively, calpain 3 is active when it is in the presence of approximately nanomolar concentrations of calcium.

Composition of Buffers:

Resuspension buffer for commercial calpains: 100 mM NaCl, 5 mM EDTA, 50 Mm TrisHCl, 5 mM β-mercaptoethanol, 40% glycerol, pH=7.8. Storage at ambient temperature. Resuspension buffer for the fluorescent peptides: the peptides are resuspended at 1 mg/ml in 100% DMSO.

Storage at 4° C., in the dark.

Reaction buffer: 100 mM NaCl, 50 mM HEPES, 10 mM DTT, 1 mM EDTA, 10% glycerol, 0.1% CHAPS, 100 ng/μl BSA, pH=7.4, filtration. Storage at ambient temperature.

7/ Eukaryotic Cell Culture:

Cell Lines Used: C2C12: murine myoblasts;

Media Used:

DMEM: Dulbecco's modified Eagle Medium (Gibco BRL)

MEM: solution of nonessential amino acids (Gibco BRL)

FCS: foetal calf serum (Gibco BRL)

Medium used for C2C12: DMEM+10% FCS

Activation of calpains: calcium chloride at a final concentration of 6 mM and ionomycin (Calbiochem) at a final concentration of 0.5 μM are added to the culture medium.

8/ Protocol for Transfection of Expression Vectors into Eukaryotic Cells:

Materials:

The DNA of the various plasmids is prepared according to the QIAGEN EndoFree protocol (ref. 12362) Plasmids used: pECFP-N1 (Clontech); pEYFP-C1 (Clontech)

Methods:

1st day:

The desired cells are trypsinized and then the wells are seeded in such a way that the cells are at 50-80% confluency the following day.

2nd day:

94 μl of medium without foetal calf serum are placed in a first sterile polystyrene tube (T1).

6 μl of FUGENE 6 buffer (Boeringher Mannheim) are added to each tube T1.

Incubation is carried out for 5 min at ambient temperature.

1 to 2 μg of EndoFree plasmid are placed in a second sterile polystyrene tube (T2), at the bottom of the tube.

At the end of the 5 min of incubation, the FUGENE 6 (T1) buffer+medium are placed dropwise in the tube T2 containing the vector.

Incubation is carried out for 30 min at ambient temperature with no agitation.

The medium in the wells is changed.

After 30 min, the transfection mixture is added drop-wise to each well, distributing the drops over the entire surface of the well.

Allow the cells to grow at 37° C./7% CO₂.

Comment: For each transfection, a well which has not been in contact with the FUGENE 6 is provided (cell control), as is a well in which the cells will have been in contact with the FUGENE 6 only (control for toxicity of the FUGENE 6).

9/ Cell Lysis Protocol:

Principle: The animal cells, which may or may not be transfected, are lysed for the purpose of preparing protein extracts the properties of which may be studied.

Method:

The culture medium is removed from the wells and the wells are washed with PBS (phosphate buffered Dulbecco solution (without calcium, or magnesium, or sodium bicarbonate)—Gibco BRL).

The cells are harvested and centrifuged at 500 g for 10 min at 4° C.

The supernatant is removed and the cells are taken up in the lysis buffer (50 mM HEPES, 1 mM DTT, 0.1 mM EDTA, 0.1% CHAPS, pH=7.4) in a proportion of 700 μl of lysis buffer per well.

The buffer is allowed to act at 4° C. for 5 minutes. Centrifugation is carried out at 10 000 g/10 minutes/4° C.

The supernatant is recovered and stored at −20° C.

10-Tissue Lysis Protocol:

Principle: The muscles are ground for the purpose of preparing protein extracts the properties of which may then be studied.

Material: Quadriceps of normal mice and of calpain 3-deficient mice.

Method:

The muscles are ground in liquid nitrogen. The ground material is resuspended in the lysis buffer (cf. composition in the cell lysis protocol above) in a proportion of 19 μl of lysis buffer per milligram of ground tissue.

The buffer is allowed to act for 10 minutes in ice. Centrifugation is carried out at 10 000 g for 10 minutes at 4° C.

The supernatant is recovered and stored at −20° C.

### B/ Assay of Specificity of the “Autolytic Sites” Peptide Cleavage by Calpain 3

Before verifying whether calpain 3 can cleave the peptides corresponding to its autolytic sites, a first assay consisted in verifying that these peptides (site 1, site 2, site 3) could not be cleaved by other proteases, in particular by ubiquitous calpains (see FIG. 2). Calpains 1 and 2 have no cleavage activity on each of the autolytic sites since the control, “peptide without enzyme” curves can be superimposed on the “peptide+enzyme” curves. Similar results were obtained with another protease, caspase 3.

It is known that calpain 3 has greater stability in muscle cells due to the presence of titin. For the purpose of proving that calpain 3 could cleave its own autolytic sites, it was therefore overexpressed in C2C12 cells by transfecting these cells with a plasmid encoding calpain 3. A control transfection with pECFP was carried out at the same time as the transfection with the plasmid encoding calpain 3. The efficiency of transfection on the C2C12s is less than 10%. The proteins were then extracted and their activity was assayed on the autolytic sites (FIG. 3).

The curves representing the activity of transfected or untransfected C2C12 extracts on sites 1 and 2 show a slight increase in fluorescence, which might mean that calpain 3 can cleave these sites. Calpain 3 is a protein expressed in muscle, therefore the basal activity observed for the untransfected cell extracts is normal. However, no difference can be detected between transfected and untransfected cells for sites 1 and 2. On the other hand, on site 3, the cleavage activity is greater in the transfected cells. The difference is minimal, but it is coherent with the low percentage of transfected cells.

The system is then assayed on extracts of cell cultures deficient or not deficient in calpain 3. To do this, mice deficient for the calpain 3 gene, for which myogenic cell cultures have been derived [Richard, 2000], are used. Extracts of cultures of calpain 3-deficient muscle cells (−/−cells) can therefore be compared with extracts of cultures of normal muscle cells (+/+cells). Initially, the assay was carried out on undifferentiated cells (myoblasts) (FIG. 4).

These experiments made it possible to show that a low cleavage activity could be detected on site 1. The difference in activity between the +/+and −/−cells is, however, very small. No cleavage activity could be detected on site 2. Cleavage activity is detected on site 3, but no difference in activity is visible between +/+cells and −/−cells.

In order to try to demonstrate a greater difference in activity between +/+extracts and −/−extracts, the same assays were carried out on extracts of muscle cells differentiated into myotubes (FIG. 5). Specifically, calpain 3 is theoretically expressed more in myotubes than in myoblasts.

This experiment makes it possible to show that the cleavage activity site 3 in the extracts of −/−cells is greater than in the extracts of +/+cells, which is surprising since the calpain 3 deficiency is the only difference between the +/+cells and the −/−cells. A decrease in activity in the calpain 3-deficient cells will therefore be more probable.

In order to try to demonstrate a greater difference in activity between extracts of +/+cells than of −/−cells, the same reaction was carried out over 6 h instead of 1 h (FIG. 6).

The cleavage activity on sites 1 and 2 is greater in extracts of +/+cells and in extracts of −/−cells. The increase in activity on site 3 is here confirmed when the reaction is carried out over 6 h. The activity is greater in extracts of −/−cells than in extracts of +/+cells. These experiments make it possible to show that, when the assay is carried out over six hours, the cleavage activity on the 3 sites makes it possible to differentiate the +/+and −/−cell types.

Activity assays were also carried out on lysates of muscles from mice deficient or not deficient in calpain 3. The assays were carried out using quadriceps lysates (FIG. 7).

The activity of cleavage of sites 1 and 2 by the +/+ tissue extracts is greater than that of the −/−tissue extracts. The activity of cleavage site 3 by the −/−tissue extracts is greater than that of the +/+tissue extracts. These experiments made it possible to show that the results obtained using tissue extracts are similar to those obtained using cell extracts. In particular, a greater activity on site 3 with extracts in which calpain 3 is not present is confirmed. This activity is therefore thought to be probably due to another protease, the activity of which is activated in the absence of calpain 3.

### II/ ASSAY OF ACTIVITY OF CALPAIN 3 IN CELLS

### A/ Materials and Methods

Cloning of Oligonucleotides into the Expression Vector pTOM

Principle: Two complementary oligonucleotides are brought together so as to form double-stranded sequences corresponding to the autolytic sites of calpain 3. The oligonucleotides are chosen in such a way that pairing thereof forms, at each end, restriction sites. These fragments are cloned into the expression vector pTOM predigested with restriction enzymes.

Materials:

Oligonucleotides: site 1, site 2, site 3

Restriction enzymes: BamH1, BspE1, SmaI (BioLabs); Ecl136II (Fermentas).

Bacterial strains used: SCS110: dam- Str^{R} bacteria (Stratagene); XL1-Blue (Stratagene).

Plasmid vector: pTOM (Genethon) (FIG. 12).

Methods:

Hybridization of the Two Complementary Oligonucleotides:

The single-stranded complementary oligonucleotides are brought into contact with one another, each at a final concentration of 20 ng/μl, in SYBR Green PCR Buffer (Applied Biosystems). The hybridization is carried out in the ABI Prism 7700 device (Applied Biosystems) according to the following conditions: 95° C./1 min→90° C./30 sec →85° C./30 sec →80° C./30 sec →75° C./30 sec →70° C./30 sec →65° C./30 sec →60° C./30 sec →55° C./30 sec. The development of the pairing is followed over time using the Sequence Detector 1.6.3 software.

Digestion of the Vector pTOM:

The vector pTOM is digested with the two enzymes BamH1 and BspE1 at 37° C. for 2 h for each enzyme.

Ligation of the Oligonucleotide in pTOM:

In the ligation mixture, the ratio between the amount of insert and the amount of vector is 3 (in mole). The ligation reaction is carried out in the presence of T4 DNA ligase (BioLabs) overnight at 16° C.

Protocol for Preparing Electrocompetent Bacteria:

15 ml of LB+ selection agent are seeded with 10 μl of bacteria stored at −80° C. in 20% glycerol. Incubation is carried out for 8 hours at 37° C. with shaking (approximately 300 rpm). 200 ml of LB+ possible selection agent are seeded with 2 ml of preculture. The bacteria are allowed to grow until OD (600 nm)=0.5.

All the material to be used must have been cooled to 4° C. beforehand.

The culture is allowed to stand in ice for 15 minutes and is then distributed in a proportion of 25 ml per tube (8 tubes).

Centrifugation is carried out at 4 000 rpm for 20 minutes at 4° C.

The supernatants are removed and the pellets are gently taken up in 200 ml of iced water (25 ml per tube -8 tubes), and centrifugation is carried out as previously.

The supernatants are removed and the pellets are taken up in 100 ml of iced water (pooling the tubes 2 by 2; 4 tubes), and centrifugation is carried out as previously.

The supernatants are carefully removed and each pellet is taken up in 5 ml of 10% glycerol (autoclaved and stored at −20° C.) and the 4 volumes are pooled in a single tube; centrifugation is carried out as previously.

The supernatants are removed, and the pellet is taken up in 400 μl of 10% glycerol. 40 μl aliquots are distributed into eppendorf, and frozen at −80° C.

Control of competent bacteria: they are transformed with a control plasmid according to the protocol described below. The titre should be greater than 10⁸ colonies per microgram of vector transformed.

Transformation Protocol:

1 μl of ligated DNA (≈0.1 ng) is added to 40 μl of electrocompetent bacteria and left in contact for 4 min in ice.

The mixture is placed in the electroporation tank (Biorad gene pulser cuvette; 0.2 cm)

Electroporation conditions: 2 500 V, 200 ohm, 25 μF. 1 ml of SOC is immediately added and allowed to stand for 30 minutes to one hour at 370° C. in order to allow expression of the gene to resistance to the antibiotic. 20 μl and 200 μl are plated out onto Petri dishes of LB + kanamycin at a final concentration of 10 μg/ml.

The bacteria are allowed to grow overnight at 37° C.

Analysis of Colonies:

The colonies are counted and subcultured in a 96-well plate in 100 μl of LB+ kanamycin at a final concentration of 10 μg/ml. To verify the presence of the plasmid in the colonies, a PCR is carried out on these colonies, either with the pair midEYFP.a and midECFP.m, or the pair formed by one of the following forward oligos:

| | |
|---|---|
| SFp94S1.a CCGGAAGTGGCACGAACATG | Each oligonucleo- |
| for site 1 | tide is specific |
| SGp94S2.a CCGGAAGTGGCGTGAGAAAT | for a cloned |
| for site 2 | double-stranded |
| SGp94S3.a CCGGAAGTGGCATTGTTCCC | fragment. They are |
| for site 3 | centred at the |
| | BspEI restriction |
| | site. |

. . . and the reverse oligonucleotide midECFP.m.

The PCR products are loaded onto a gel composed of 0.8% agarose+ethidium bromide at a final concentration of 0.5 μg/ml. To verify the presence of an insert in the plasmids, a certain number of PCR products of positive clones are sequenced with the oligonucleotide midECFP.m.

2-Eukaryotic Cell Culture:

Cell lines used: C2C12: murine myoblasts

Media used:

DMEM: Dulbecco's modified Eagle Medium (Gibco BRL)

MEM: solution of nonessential amino acids (Gibco BRL)

FCS: foetal calf serum (Gibco BRL)

Medium used for the 911 line: DMEM+ 10% FCS+ 1% MEM

Medium used for the Cos7 and C2C12 lines: DMEM+ 10% FCS

Activation of calpains: calcium chloride at a final concentration of 6 mM and ionomycin (Calbiochem) at a final concentration of 0.5 μM are added to the culture medium.

3-Protocol for Transfecting Expression Vectors into Eukaryotic Cells:

Materials:

The DNA of the various plasmids is prepared according to the QIAGEN EndoFree protocol (ref. 12362).

Plasmids used: pECFP-N1 (Clontech); pEYFP-C1 (Clontech)

Methods:

1st day:

The desired cells are trypsinized and then the wells are seeded in such a way that the cells are at 50-80% confluency the following day.

2nd day:

94 μl of medium without foetal calf serum are placed in a first sterile polystyrene tube (T1).

6 μl of FUGENE 6 buffer (Boeringher Mannheim) are added to each tube T1.

Incubation is carried out for 5 min at ambient temperature.

1 to 2 μg of EndoFree plasmid are placed in a second sterile polystyrene tube (T2), at the bottom of the tube.

At the end of the 5 min of incubation, the FUGENE 6 (T1) buffer+medium is placed dropwise in the tube T2 containing the vector.

Incubation is carried out for 30 min at ambient temperature with no agitation.

The medium in the wells is changed.

After 30 min, the transfection mixture is added drop-wise to each well, distributing the drops over the entire surface of the well.

The cells are allowed to grow at 37° C./7% CO₂.

Comment: For each transfection, a well which has not been in contact with the FUGENE 6 is provided (cell control), as is a well in which the cells will have been in contact with the FUGENE 6 only (control for toxicity of the FUGENE 6);

### B/ Results

In order to detect the protolytic activity of calpain 3 in live cells, the cleavage of its three autolytic sites are studied using the FRET phenomenon.

For the purpose of having a “FRET positive” control, i.e. a plasmid encoding an ECFP-EYFP chimeric protein which cannot be cleaved by calpains and within which a FRET phenomenon can occur, the vector pTOM was initially modified in such a way that the coding sequences for the two fluorescent proteins Enhanced-CFP (ECFP) and Enhanced-YFP (EYFP) which are on this vector are in phase. DNA fragments encoding the three auto-lytic sites of calpain 3 were then inserted between these sequences. We then verified that the chimeric protein produced could really be cleaved in vitro by ubiquitous calpains. Finally, this cleavage was studied in cells by fluorescence image analysis. Three distinct methods were used for this analysis, and in particular for that of the FRET.

1-Cloning of a Control Vector and of the Vectors Carrying the DNA Fragments Encoding the Cleavage Sites:

Construction of a Vector Encoding Two Fluorescent Proteins in Phase:

The strategy for constructing the vector carrying the two sequences encoding ECFP and EYFP in phase (called pTOMp) consisted in digesting the plasmid pTOM with two restriction enzymes having unique sites on pTOM and generating blunt-ended ends, Ecl136II and SmaI (FIG. 8). The linearized vector was purified and a reaction to ligate the vector on itself was carried out. After transformation and subculturing of positive colonies, a PCR reaction made it possible to confirm the presence of the plasmid in these colonies (FIG. 9) Approximately one third of the colonies subcultured could thus be amplified and therefore should have contained the vector pTOMp. The digestion of these PCR products with EcoRI, the restriction site for which disappeared with the elimination of the Ecl136II-SmaI fragment, made it possible to confirm the colonies subcultured indeed contained a plasmid pTOMp and not the plasmid of origin. Sequencing of the PCR products made it possible to verify that the two sequences were indeed in phase with one another.

2-Cloning of Oligonucleotides Encoding Sites of Cleavage by Calpain 3 in the Expression Vector pTOM:

In order to facilitate the FRET phenomenon between the two proteins EYFP and ECFP, glycine and serine amino acids were added on either side of the cleavage site so as to facilitate the bringing together of the two proteins, the glycines facilitating the flexibility of the chimeric protein and the serines increasing its solubility in an aqueous medium (FIG. 10). The sequence of the oligonucleotides is such that they form restriction sites for BspE1 and BamHI at each end when they are paired.

The bases marked in bold and underlined in the sequences of the oligonucleotides are bases which do not modify the protein sequence but which are different from the genomic sequence. These bases were modified so as to limit homologous primer duplex formation, which might impede the formation of double-stranded oligonucleotides. The base modification is also carried out as a function of the codon use frequency in mice.

The restriction sites used for the cloning are unique sites. The BspE1 enzyme is inactive if the cloning site is methylated. Cloning of the vector pTOM intended to receive the double-stranded oligonucleotides was therefore carried out in bacteria in which the gene encoding Dam methylase is mutated. After digestion of the vector with the BspE1 and BamH1 enzymes, ligation of the oligonucleotides and electroporation, some resistant colonies are sampled. The presence of the plasmid is verified by PCR with midECFP.m and an oligonucleotide specific for the restriction site (FIG. 11). The sequencing on some positive clones obtained by PCR made it possible to verify the presence of the inserts in pTOM, that these inserts were indeed in phase with the sequences encoding the ECFP and EYFP proteins, and that they did not comprise any mutations. Three colonies containing the three cloned plasmids are conserved. The three plasmids correspond to the three inserted DNA fragments: the three calpain 3 cleavage sites (plasmids pTOMs1, pTOMs2 and pTOMs3) (FIG. 13).

### BIBLIOGRAPHY

Sorimachi, H., Ishiura, S., Suzuki, K. (1997). “Structure and physiological function of calpains.” Biochemistry Journal 328: 721-732.

Squier, M. K. T., Miller, A. K. C., Malkinson, A. M., Cohen, J. J. (1994). “Calpain activation in apoptosis.” J. Cell. Physiol. 159: 229-237.

Kwak, K. B., Chung, S. S., Kim, O. M., Kang, M. S., Ha, D. B., Chung, C. H. (1993). “Increase in the level of m-caplain correlates with the elevated cleavage of filamin during mycogenic differentiation of embryonic muscle cell.” Biochim. Biophys. Acta 1175: 243-249.

Yamaguchi, R., Maki, M., Hatanaka, M., Sabe, H. (1994). “Unphosphorylated and tyrosine-phosphorylated forms of a focal adhesion protein, paxillin, are substrates for calpain II in vitro: Implications for the possible involvement of calpain II in mitosis-specific degradation of paxillin.” FEBS lett. 356: 114-116.

Schollmeyer, J. E. (1986). “Role of Ca2+ and Ca2+-activated protease in myoblast fusion.” Exp. Cell. Res. 162: 411-422.

Balcerzak, D., Poussard, S., Brutis, J. J., Elamrani, N., Soriano, M., Cottin, P., Ducastaing, A. (1995). “An antisense oligodeoxyribonucleotide to m-caplain inhibits myoblast fusion.” J. Cell. Scien. 108: 2077-2082.

Sorimachi, H., Imajoh-Ohmi, S., Emori, Y., Kawasaki, H., Ohno, S., Minami, Y., Suzuki, K. (1989). “Molecular cloning of a novel mammalian calcium-dependent protease distinct from both m- and mu-type. Specific expression of the mRNA in skeletal muscle.” J. Biol. Chem. 264: 20106-20111.

Richard I., Broux, O., Allamand, V., Fougerousse, F., Chiannilkulchai, N., Bourg, N., Brenguier, L., Devaud, C., Pasturaud, P., Roudaut, C., Hillaire, D., Passos-Bueno, M. R., Zatz, M., Tischfield, J. A., Fardeau, M., Jackson, C. E., Cohen, D., Beckmann, J. S. (1995). “Mutations in the proteolytic enzyme, calpain 3, cause limb-girdle muscular dystrophy type 2A.” Cell 81: 27-40.

Fardeau, M., Hillaire, D., Mignard, C., Feingold, N., Mignard, D., de Ubeda, B., Collin, H., Tomé, F. M. S., Richard, I., Beckmann, J. S. (1996). “Juvenile limb-girdle muscular dystrophy: clinical, histopathological, and genetic data from a small community living in the Reunion Island.” Brain 119: 295-308.

Sorimachi, H., Toyama-Sorimachi, N., Saido, T. C., Kawasaki, H., Sugita, H., Miyasaka, M., Arahata, K., Ishiura, S., Suzuki, K. (1993). “Muscle-specific calpain, p94, is degraded by autolysis immediately after translation, resulting in disappearance from muscle.” J. Biol. Chem. 268: 10593-10605.

Sorimachi, H., Kimbara, K., Kimura, S., Takahashi, M., Ishiura, S., Sasagawa, N., Sorimachi, N., Shimada, H., Tagawa, K., Maruyama, K., Suzuki, K. (1995). “Muscle-specific calpain, p94, responsible for limb-girdle muscular dystrophy type 2A, associates with connection through IS2, a p94-specific sequence.” J. Biol. Chem. 270: 31158-31162.

Guttman et al. Methods in molecular biology vol 144, ch 18

Förster, T. (1948). “Intermolecular energy migration and fluorescence.” Ann. Phys. (Leipzig) 2: 55-75.

Prasher, D. C., Eckenrode, V. K., Ward, W. W., Prendergast, F. G., Cormier, M. J. (1992). “Primary structure of the Aequorea Victoria green-fluorescent protein.” Gene 111: 229-33.

Ellenberg J, Lippincott-Schwartz J, Presley J F. (1999). “Dual-colour imaging with GFP variants.” Trends cell Biol. 2: 52-6.

Pollock, B., Heim, R. (1999). “Using GFP as FRET-based applications.” Trend Cell Biol. 2: 57-60.

Richard, I., Roudaut, C., Marchand, S., Baghdiguian, S., Herasse, M., Stockholm, D., Ono, Y., Suel, L., Bourg, N., Sorimachi, H., Lefranc, G., Fardeau, M., Sébille, A., Beckmann, J. S. (2000). “Loss of calpain 3 proteolytic activity leads to muscular dystrophy and to perturbation of the IkBa/NF-kB pathway in mice.” J. Cell. Biol. 151: 1583-1590.

Vanderklish, P. W., Krushel, L. A., Holst, B. H., Gaily, J. A., Crossin, K. L., Edelman, G. M. (2000). Marking synaptic activity in dendritic spines with a calpain substrate exhibiting fluorescence resonance energy transfer. Proc Natl Acad Sci USA.; 5: 2253-8.

1. Peptide coupled to at least one fluorogenic or colorigenic reporter molecule, characterized in that it contains at least one amino acid sequence able to be cleaved by calpain 3 or an isoform of calpain 3.

2. Peptide according to claim 1, characterized in that it contains at least one autolytic site of calpain 3 or of an isoform of calpain 3, the number of amino acids of which is less than 10.

3. Peptide according to claim 2, characterized in that the amino acid sequence of the autolytic sites of calpain 3 is chosen from the following sequences: NMTYGTS (SEQ ID1), NMDNSLL (SEQ ID2), PVQYETR (SEQ ID3).

4. Peptide according to claim 2, characterized in that the amino acid sequence of the autolytlc site of calpain-3 is chosen from the following sequences VAPRTA AEPRSP (SEQ ID4), QSKATE AGGGNP (SEQ ID5), and the following murine sequences VAPRTG AEPRSP (SEQ ID6), QGKTTE AGGGHP (SEQ ID7).

5. Peptide according to claim 2, characterized in that the amino acid sequence of the autolytic site originates from an isoform of calpain 3 named Lp82, and is chosen from the following amino acid sequences: NPYLLPGFFC (SEQ ID18) and TISVDRPVP (SEQ ID19).

6. Peptide according to claim 1, characterized in that the sequence which can be cleaved by calpain 3 or an isoform of calpain 3 originates from a substrate protein and is chosen from the following sequences: REVTIPPKYRELL (SEQ ID10), KEGTIPPEYRKLL (SEQ ID11), PVSREEKPTSAPSS (SEQ ID12), PVSREEKPSSAPSS (SEQ ID13), KSTVLQQQYNR (SEQ ID14).

7. Peptide according to claim 1, characterized in that the peptide is obtained by screening a peptide library with calpain 3 or an isoform of calpain 3.

8. Peptide according to claim 1, characterized in that it has, at each of its ends, a synthetic fluorogenic reporter molecule, respectively MCA (donor molecule) and Dnp (acceptor molecule).

9. Peptide according to claim 1, characterized in that the reporter molecule is a protein.

10. Peptide according to claim 9, characterized in that it has, at each of its ends, a mutated GFP.

11. Peptide according to claim 10, characterized in that the mutated GFPs are CFP and YFP, respectively.

12. DNA sequence encoding the peptide which is the subject of claim 1.

13. Vector containing the DNA sequence which is the subject of claim 12 and a promoter for reducing expression of the DNA sequence in a host cell.

14. Host cell transformed with the vector which is the subject of claim 13.

15. Method for detecting, in vitro, the activity of calpain 3 or an isoform of calpain 3 in a biological sample, according to which:
in a first step, said biological sample is brought into contact with a peptide which is the subject of claim 1,
in a second step, the presence or absence of cleavage of said peptide by calpain 3 or an isoform of calpain 3 is detected by measuring the intensity of the calorimetric or fluorometric reaction.

16. Method according to claim 15, characterized in that the first step consists in transfecting host cells with the vector which is subject to claim 13.

17. Method according to claim 15, characterized in that the first step consists in bringing the peptide which is the subject of claim 1 into contact with a cell extract or the tissue section.

18. Method according to claim 15, characterized in that the peptide has, at each of its ends, a fluoro-genic donor molecule and a fluorogenic acceptor molecule, respectively, the intensity of the fluoro-genic reaction being determined by FRET.

19. Use of the method which is the subject of claim 15, for the in vitro diagnosis of LGMD 2A.

20. Method for screening for substances which activate or inhibit calpain 3 or an isoform of calpain 3, said method consisting:
in preparing a biological sample treated with said substance,
then in bringing said sample thus treated into contact with the peptide of claim 1,
and in detecting the presence or absence of a calorimetric or fluorometric reaction, indicating respectively the presence or absence of a substance which activates or inhibits calpain 3 or an isoform of calpain 3.

21. Method according to claim 20, characterized in that the peptide has, at each of its ends, a fluorogenic donor molecule and a fluorogenic acceptor molecule, respectively, the intensity of the fluorogenic reaction being determined by FRET.

22. Method for screening for substances which activate or inhibit calpain 3 or an isoform of calpain 3, said method consisting:
in preparing a biological sample containing the peptide which is the subject of claim 1,
then in bringing said sample into contact with the substance to be identified,
and in detecting the presence or absence of a calorimetric or fluorometric reaction, indicating respectively the presence or absence of a substance which activates or inhibits calpain 3 or an isoform of calpain 3.

23. Method according to claim 22, characterized in that the peptide has, at each of its ends, a fluorogenic donor molecule and a fluorogenic acceptor molecule, respectively, and it consists in:
a/ preparing a biological sample containing said peptide,
b/ measuring the amount of FRET in the absence of the substance which activates or inhibits calpain 3 or an isoform of calpain 3,
c/ bringing the biological sample containing the peptide into contact with the substance which activates or inhibits calpain 3 or an isoform of calpain 3
d/ measuring the amount of FRET in the presence of the substance which activates or inhibits calpain 3 or an isoform of calpain 3,
e/ drawing the conclusion of the presence: of an activating substance if the amount of FRET measured in b/ is greater than the amount of FRET measured in d/ ;
or of an inhibiting substance if the amount of FRET measured in b/ is equal to the amount of FRET measured in d/ .

24. Method for analysing the efficiency of transfer of the calpain 3 gene, consisting:
first of all in transfecting animal or human cells with the calpain 3 gene,
then in bringing the transfected cells into contact with the peptide which is the subject of claim 1 in vitro,
and in measuring the intensity of the calorimetric or fluorometric reaction.

25. Method according to claim 24, characterized in that the peptide has, at each of its ends, a fluoro-genic donor molecule and a fluorogenic acceptor molecule, respectively, the intensity of the fluoro-genic reaction being determined by FRET.
